(19) United States
(12) Patent Application Publication
(10) Pub. No.: US 2024/0041942 A1
(43) Pub. Date: Feb. 8, 2024

(54) JOINT RELATED COMPOSITION AND METHODS OF USE

(71) Applicant: Jon Gregory, China Springs, TX (US)

(72) Inventor: Jon Gregory, China Springs, TX (US)

(21) Appl. No.: 18/229,599

(22) Filed: Aug. 2, 2023

Related U.S. Application Data
(60) Provisional application No. 63/395,051, filed on Aug. 4, 2022.

(51) Int. Cl. A61K 35/57; A61K 31/12; A61K 35/618; A61K 36/54; A61K 36/48; A61K 36/82; A61K 38/39; A61P 19/02; A61K 9/00

(52) U.S. Cl. CPC A61K 35/57; A61K 31/12; A61K 35/618; A61K 36/54; A61K 36/48; A61K 36/82; A61K 38/39; A61P 19/02; A61K 9/0056

(57) ABSTRACT

A joint related composition includes eggshell membrane in an amount of 7% to 15.5%; perna canaliculus in an amount of 50% to 80%; curcumin in an amount of 1% to 5%; avocado or soybean unsaponifiables in an amount of 0.5% to 5%; green tea leaf extract in an amount of 5% to 15%; or collagen in an amount of 0.2% to 3%.

### CROSS-REFERENCE TO RELATED APPLICATION(S)

This application claims benefit of U.S. Provisional Application No. 63/395,051, filed Aug. 4, 2022, which is incorporated herein by reference in its entirety.

### BACKGROUND

Increasingly, pets, such as cats and dogs, are becoming an integral part of the family. Such increased care and attention has led to improvements in lifestyle, food quality, and healthcare. As a result, several studies have shown that pets are living longer lives, in some cases close to 10% longer lives.

However, with longer lives, pets are increasingly suffering from age-related ailments. In one example, joint health suffers as pets age. Joint related issues are particularly acute in larger canine breeds. Much in line with the increased care and affection, owners are seeking to alleviate pain and suffering associated with such joint issues.

As such, improved supplements and methods for addressing joint related issues in pets, such as canines, would be desirable.

### DETAILED DESCRIPTION

In an exemplary embodiment, the joint related composition for pets, such as canines or felines, includes at least three of the ingredients: eggshell membrane, perna canaliculus, curcumin, avocado or soybean unsaponifiables, green tea leaf extract, or collagen. In an example, the joint related composition includes eggshell membrane, green tea leaf extract, and collagen. In another example, the joint related composition includes perna canaliculus, green tea leaf extract, and collagen. In a particular example, the composition includes each of eggshell membrane, perna canaliculus, curcumin, avocado or soybean unsaponifiables, green tea leaf extract, and collagen. In an example, the joint related composition is in the form of a powder and can be added into the food of a pet, such as a canine or feline. Alternatively, the composition can be in the form of a pill or tablet.

The joint related composition can include eggshell membrane. Eggshell membrane contains a number of natural substances including collagen, elastin, desmosine and isodesmosine, transforming growth factor-b, or glycosaminoglycans. Collagen can support cartilage and connective tissue strength and elasticity. Elastin can support skin, cardiovascular, cartilage, and spinal health. Desmosine and isodesmosine are amino acids responsible for elastin's elastic properties. Glycosaminoglycans include glucosamine, chondroitin, or hyaluronic acid.

In example, the joint related composition includes eggshell membrane in an amount of 7% to 15.5%. For example, the eggshell membrane can be included in the joint related composition in an amount of 9% to 13.5%, such as an amount of 11% to 13%.

The composition may also contain anti-inflammatory or natural joint health promoting compounds. For example, the composition can include perna canaliculus, Boswellia serrata, turmeric (Curcuma longa), stinging nettle (Urtica dioica), or Kalmegh, cat's claw (Uncaria tomentosa).

For example, the joint related composition can include perna canaliculus in amount of 50% to 80%. For example, the perna canaliculus can be included in an amount of 60% to 80%, such as an amount of 65% to 75%.

The joint related composition can, for example, include curcumin in amount of 1% to 5%. For example, the curcumin can be included in amount 2% to 4.5%, such as in an amount of 3% to 4%. In an example, the curcumin can be derived from turmeric.

In a further example, the joint related composition can include avocado or soybean unsaponifiables. Avocado or soybean unsaponifiables are natural vegetable extracts made from avocado or soybean oils, consisting of the leftover fraction that does not saponificate into soap. In an example, the avocado or soybean unsaponifiables is composed of one third avocado and two thirds soybean unsaponifiables. Avocado or soybean unsaponifiables can include phytosterols, such as sitosterol, campesterol, and stigmasterol. Avocado or soybean unsaponifiables can further includes fat-soluble vitamins, sterols, triterpene alcohols, or furan fatty acids. Avocado or soybean unsaponifiables can contribute to biological activity in articular chondrocytes.

Avocado or soybean unsaponifiables may improve joint function and alleviate joint pain by helping reduce erosion of cartilage. It may increase the synthesis of collagen and proteoglycan and help preserve hydroxyproline, which promotes collagen stability. Avocado or soybean unsaponifiables may decrease the synthesis of fibronectin and inhibit the release of substances such as metalloproteinases and proinflammatory cytokines.

In an example, the joint related composition can include avocado or soybean unsaponifiables in about 0.5% to 5%. For example, the avocado or soybean unsaponifiables can be included in the joint related composition in an amount of 1% to 4%, such as in an amount of 2% to 3.5%.

In an additional example, the joint related composition can include tea extracts, such as green tea extracts. Tea (Camellia sinensis), especially green tea, has catechins as an active ingredient. For example, green tea extract can include epigallocatechin gallate (EGCG), epicatechin (EC), epigallocatechin (EGC), and epicatechin gallate (ECG). Green tea and EGCG may protect against cartilage loss and reduce the progression of osteoarthritis. Green tea catechins (GTCs) may also assist with re-establishing homeostasis of skeletal muscle cells and attenuating muscle mass loss.

EGCG may also have anabolic effects on osteoarthritis. EGCG may stimulate the IL-1β-induced expression of transforming growth factor (TGF)-β1, TGF-β2, and its receptors TGF-βR1 and TGF-βRII in articular chondrocytes, resulting in enhanced type II collagen and aggrecan core protein synthesis in articular chondrocytes. EGCG supplementation may stimulate chondrocyte growth and the synthesis of cartilage extracellular matrix. Further, EGCG with eggshell membrane may synergistically stimulate chondrogenic regeneration.

Green tea extract can be included in the joint related composition in amount of 5% to 15%. For example, the green tea leaf extract can be included in the composition in an amount of 7% to 12%, such as an amount of 8% to 11%.

The joint related composition can further include collagen. The collagen can, for example, be an undenatured type II collagen, such as undenatured type II chicken collagen. When consumed, the undenatured type II collagen may activate immune cells, for example, transforming naive T-cells into T regulatory (Treg) cells that target type II collagen. When the Treg cells recognize type II collagen in joint cartilage, they may secrete anti-inflammatory mediators (cytokines), including the transforming growth factor-beta (TGF-beta), interleukin 4 (IL-4) and interleukin 10 (IL-1β), which may reduce joint inflammation and promote cartilage repair. The collagen can be included in amount of 0.2% to 3%, such as in an amount of 0.5% to 2.5% or in an amount of 0.8% to 1.8%.

In an example, the joint related composition is in the form of a powder, such as a scoopable powder that can be mixed with food or treats supplied to the pet. Alternatively, the composition can be in the form of a pill or tablet. In the form of a tablet, the composition can further include fillers designed to provide structural integrity to the tablet. In a further example, the joint related composition can form a chewable treat.

The composition can include anti-caking agents. For example, the composition can include silicon dioxide.

In an example, the composition is free of fillers. Alternatively, the composition can include fillers such as a bone meal, food starches, modified food starches, or fibrous fillers such as vegetable fibers or inulin, among others.

In a further example, the composition can be free of flavorings. Alternatively, the composition can include flavorings such as salt, dried animal stock, tallow, or dried vegetable stock, among other flavorings.

Further, the composition can be free of a colorant. Alternatively, the composition can include colorants, such as food dyes.

The composition can be free of preservatives. Alternatively, the composition can include preservatives such as oxidation blocker systems, tocopherols, or ascorbic acid, among others.

Except for components of the eggshell membrane, the composition can be free added glucosamine, chondroitin, or hyaluronic acid. Further, the composition can be free of methyl sulfonyl methane. Alternatively, the composition can include such ingredients.

In a particular example, the joint related composition includes eggshell membrane in an amount of 7% to 15.5%, green tea leaf extract in an amount of 5% to 15%, and collagen in an amount of 0.2% to 3%. In another example, the joint related composition includes perna canaliculus in an amount of 50% to 80%, green tea leaf extract in amount of 5% to 15%, and collagen in the amount of 0.2% to 3%.

In a particular example, the joint related composition consists essentially of eggshell membrane in an amount of 7% to 15.5%, perna canaliculus in amount of 50% to 80%, curcumin in an amount of 1% to 5%, avocado or soybean unsaponifiables in amount of 0.5% to 5%, green tea leaf extract in amount of 5% to 15%, and collagen in amount of 0.2% to 3%. Each of the ingredients can be blended together to form a powder.

Canines and felines can be treated based on weight using the compositions described above. For example, a pet can be weighed and provided the composition in an amount of 900 mg to 1200 mg for each increment of 20 pounds in weight. In other words, a canine weighing less than 20 pounds can be provided with between 900 and 1200 mg of the composition, and a pet weighing between 20 pounds and 40 pounds can be provided with between 1800 mg and 2400 mg of the composition. In a further example, at a weight between 60 pounds and 80 pounds, a pet can be provided with the composition in amount of 3600 mg to 4800 mg. Canines weighing over 80 pounds can be provided with up to five times an amount between 900 mg and 1200 mg of the composition.

Embodiments of the example compositions provide advantages and benefits when used with pets, such as canines or felines. In particular, the ingredients in the stated compositional ranges synergistically provide benefits to pets suffering from joint related conditions.

### EXAMPLES

### Example 1

A Pyrenees canine weighing 87 pounds exhibits signs of a joint condition. The canine has difficulty walking and shows signs of pain or discomfort when moving. The canine is fed between 4500 mg and 6000 mg of a composition including eggshell membrane in an amount of 12.6%, perna canaliculus in an amount of 70.2%, curcumin in an amount of 3.7%, avocado or soybean unsaponifiables in amount of 2.8%, green tea leaf extract in an amount of 9.4%, and collagen in an amount of 1.2% with each meal.

After three weeks of supplementation, the Pyrenees canine exhibits improved mobility and is able to go on walks with the owner and navigate stairs. Furthermore, the canine appears happier and more vivacious around its owner.

### Example 2

A Dalmatian canine weighing 53 pounds exhibits signs of a joint pain. The canine has difficulty climbing stairs and shows decreased activity. The canine is fed 3000 mg of a composition including eggshell membrane in an amount of 12.6%, perna canaliculus in an amount of 70.2%, curcumin in an amount of 3.7%, avocado or soybean unsaponifiables in amount of 2.8%, green tea leaf extract in an amount of 9.4%, and collagen in an amount of 1.2% with each meal.

After a month of supplementation, the owners report a significant change in activity and the ability to climb stairs.

### Example 3

A Samoyed canine weighing 36 pounds exhibits decreased activity and has trouble jumping onto furniture. The canine shows signs of pain or discomfort when moving. The canine is fed between 2000 mg of a composition including eggshell membrane in an amount of 12.6%, perna canaliculus in an amount of 70.2%, curcumin in an amount of 3.7%, avocado or soybean unsaponifiables in amount of 2.8%, green tea leaf extract in an amount of 9.4%, and collagen in an amount of 1.2% with each meal.

After two months of supplementation, the owner reports increased activity and the canine jumping on the furniture as it had prior to the problem.

In a first aspect, a joint related composition consisting essentially of eggshell membrane in an amount of 7% to 15.5%; perna canaliculus in an amount of 50% to 80%; curcumin in an amount of 1% to 5%; avocado or soybean unsaponifiables in an amount of 0.5% to 5%; green tea leaf extract in an amount of 5% to 15%; and collagen in an amount of 0.2% to 3%.

In an example of the first aspect, the collagen is undenatured type II collagen. In an example, the collagen is chicken collagen.

In another example of the first aspect and the above examples, the collagen is included in an amount of 0.5% to 2.5%. For example, the collagen is included in an amount of 0.8% to 1.8%.

In a further example of the first aspect and the above examples, the eggshell membrane is included in an amount of 9% to 13.5%. For example, the eggshell membrane is included in an amount of 11% to 13%.

In an additional example of the first aspect and the above examples, the perna canaliculus is included in an amount of 60% to 80%. For example, the perna canaliculus is included in an amount of 65% to 75%.

In another example of the first aspect and the above examples, the curcumin is included in an amount of 2% to 4.5%. For example, the curcumin is included in an amount of 3% to 4%.

In a further example of the first aspect and the above examples, the avocado or soybean unsaponifiables is included in an amount of 1% to 4%. For example, the avocado or soybean unsaponifiables is included in an amount of 2% to 3.5%.

In an addition example of the first aspect and the above examples, the green tea leaf extract is included in an amount of 7% to 12%. For example, the green tea lead extract is included in an amount of 8% to 11%.

In another example of the first aspect and the above examples, the composition is in the form of a powder. In a further example of the first aspect and the above examples, the composition further includes a filler. In an additional example of the first aspect and the above examples, the composition further includes a flavoring. In another example of the first aspect and the above examples, the composition further includes a colorant. In a further example of the first aspect and the above examples, the composition further includes a preservative.

In a second aspect, a method of providing supplements to a canine includes weighing the canine and providing the composition of any of the above aspects and examples to the canine in the amount of 900 mg to 1200 mg for each increment of 20 pounds in weight.

In a third aspect, a joint related composition includes eggshell membrane in an amount of 7% to 15.5%; green tea leaf extract in an amount of 5% to 15%; and collagen in an amount of 0.2% to 3%.

In an example of the third aspect, the collagen is undenatured type II collagen. For example, the collagen is chicken collagen.

In another example of the third aspect and the above examples, the collagen is included in an amount of 0.5% to 2.5%. For example, the collagen is included in an amount of 0.8% to 1.8%.

In a further example of the third aspect and the above examples, the eggshell membrane is included in an amount of 9% to 13.5%. For example, the eggshell membrane is included in an amount of 11% to 13%.

In an additional example of the third aspect and the above examples, the composition further includes perna canaliculus in an amount of 50% to 80%. For example, the perna canaliculus is included in an amount of 60% to 80% or the perna canaliculus is included in an amount of 65% to 75%.

In another example of the third aspect and the above examples, the composition further includes curcumin in an amount of 1% to 5%. For example, the curcumin is included in an amount of 2% to 4.5%. In an example, the curcumin is included in an amount of 3% to 4%.

In a further example of the third aspect and the above examples, the composition further includes avocado or soybean unsaponifiables in an amount of 0.5% to 5%. For example, the avocado or soybean unsaponifiables is included in an amount of 1% to 4%. In an example, the avocado or soybean unsaponifiables is included in an amount of 2% to 3.5%.

In an additional example of the third aspect and the above examples, the green tea leaf extract is included in an amount of 7% to 12%. For example, the green tea lead extract is included in an amount of 8% to 11%.

In another example of the third aspect and the above examples, the composition is in the form of a powder. In a further example of the third aspect and the above examples, the composition further includes a filler. In an additional example of the third aspect and the above examples, the composition further includes a flavoring. In another example of the third aspect and the above examples, the composition further includes a colorant. In a further example of the third aspect and the above examples, the composition further includes a preservative.

In a fourth aspect, a joint related composition includes perna canaliculus in an amount of 50% to 80%; green tea leaf extract in an amount of 5% to 15%; and collagen in an amount of 0.2% to 3%.

In an example of the fourth aspect, the collagen is undenatured type II collagen. For example, the collagen is chicken collagen.

In another example of the fourth aspect and the above examples, the collagen is included in an amount of 0.5% to 2.5%. For example, the collagen is included in an amount of 0.8% to 1.8%.

In a further example of the fourth aspect and the above examples, the composition further includes eggshell membrane in an amount of 7% to 15.5%. For example, the eggshell membrane is included in an amount of 9% to 13.5%. In another example, the eggshell membrane is included in an amount of 11% to 13%.

In an additional example of the fourth aspect and the above examples, the perna canaliculus is included in an amount of 60% to 80%. For example, the perna canaliculus is included in an amount of 65% to 75%.

In another example of the fourth aspect and the above examples, the composition further includes curcumin in an amount of 1% to 5%. For example, the curcumin is included in an amount of 2% to 4.5%. In another example, the curcumin is included in an amount of 3% to 4%.

In a further example of the fourth aspect and the above examples, the composition further includes avocado or soybean unsaponifiables in an amount of 0.5% to 5%. For example, the avocado or soybean unsaponifiables is included in an amount of 1% to 4%. In another example, the avocado or soybean unsaponifiables is included in an amount of 2% to 3.5%.

In an additional example of the fourth aspect and the above examples, the green tea leaf extract is included in an amount of 7% to 12%. For example, the green tea lead extract is included in an amount of 8% to 11%.

In another example of the fourth aspect and the above examples, the composition is in the form of a powder. In a further example of the fourth aspect and the above examples, the composition further includes a filler. In an additional example of the fourth aspect and the above examples, the composition further includes a flavoring. In another example of the fourth aspect and the above examples, the composition further includes a colorant. In a further example of the fourth aspect and the above examples, the composition further includes a preservative.

In a fifth aspect, a method of providing supplements to a canine includes weighing the canine and providing the composition of any of the above aspects and examples to the canine in the amount of 900 mg to 1200 mg for each increment of 20 pounds in weight.

As used herein, percentages are expressed as weight percentages and are calculated based on a percentage of the total weight of the active ingredients, not including fillers, colorants, preservatives, or other additives.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A joint related composition consisting essentially of:
eggshell membrane in an amount of 7% to 15.5%;
perna canaliculus in an amount of 50% to 80%;
curcumin in an amount of 1% to 5%;
avocado or soybean unsaponifiables in an amount of 0.5% to 5%;
green tea leaf extract in an amount of 5% to 15%; and
collagen in an amount of 0.2% to 3%.

2. The composition of claim 1, wherein the collagen is undenatured type II collagen.

3. The composition of claim 2, wherein the collagen is chicken collagen.

4. The composition of claim 1, wherein the collagen is included in an amount of 0.5% to 2.5%.

5. The composition of claim 4, wherein the collagen is included in an amount of 0.8% to 1.8%.

6. The composition of claim 1, wherein the eggshell membrane is included in an amount of 9% to 13.5%.

7. The composition of claim 6, wherein the eggshell membrane is included in an amount of 11% to 13%.

8. The composition of claim 1, wherein the perna canaliculus is included in an amount of 60% to 80%.

9. The composition of claim 8, wherein the perna canaliculus is included in an amount of 65% to 75%.

10. The composition of claim 1, wherein the curcumin is included in an amount of 2% to 4.5%.

11. The composition of claim 10, wherein the curcumin is included in an amount of 3% to 4%.

12. The composition of claim 1, wherein the avocado or soybean unsaponifiables is included in an amount of 1% to 4%.

13. The composition of claim 12, wherein the avocado or soybean unsaponifiables is included in an amount of 2% to 3.5%.

14. The composition of claim 1, wherein the green tea leaf extract is included in an amount of 7% to 12%.

15. The composition of claim 14, wherein the green tea lead extract is included in an amount of 8% to 11%.

16. The composition of claim 1, wherein the composition is in the form of a powder.

17. The composition of claim 1, further comprising a filler.

18. The composition of claim 1, further comprising a flavoring.

19. The composition of claim 1, further comprising a colorant.

20. The composition of claim 1, further comprising a preservative.

21.-68. (canceled)
